(19) United States
(12) Patent Application Publication
(10) Pub. No.: US 2015/0047976 A1
(43) Pub. Date: Feb. 19, 2015

(54) ANALYTICAL TEST STRIP HAVING CANTILEVERED CONTACTS

(71) Applicant: Cilag GmbH International, Zug (CH)

(72) Inventor: Steven SETFORD, Inverness (GB)

(73) Assignee: Cilag GmbH International, Zug (CH)

(21) Appl. No.: 13/968,975

(22) Filed: Aug. 16, 2013

(51) Int. Cl. G01N 27/327; C12Q 1/00

(52) U.S. Cl. CPC G01N 27/327; C12Q 1/006
USPC 204/403.01; 156/182

(57) ABSTRACT

A biosensor for use as an analytical test strip is formed as a main body having a first electrode facing in a first direction, a second electrode facing in a second direction, a pair of spacers disposed between the first and second electrodes, wherein the first electrode extends from the main body of the biosensor at a first angle and the second electrode extends from the main body at a second angle transverse to the first angle.

### TECHNICAL FIELD

The present disclosure relates to structures, functions, and fabrication methods for a biosensor, and more particularly a test strip used for analyte detection.

### BACKGROUND

Blood glucose measurement systems typically comprise an analyte meter that is configured to receive a biosensor, usually in the form of an analytical test strip. A user may obtain a small sample of blood typically by a fingertip skin prick and then may apply the sample to the test strip to begin a blood analyte assay. Because many of these measurement systems are portable, and testing can be completed in a short amount of time, patients are able to use such devices in the normal course of their daily lives without significant interruption to their personal routines. A person with diabetes may measure their blood glucose levels several times a day as a part of a self management process to ensure glycemic control of their blood glucose within a target range. A failure to maintain target glycemic control can result in serious diabetes-related complications including cardiovascular disease, kidney disease, nerve damage and blindness.

Analyte detection assays find use in a variety of applications, including clinical laboratory testing, home testing, etc., where the results of such testing play a prominent role in diagnosis and management in a variety of disease conditions. Analytes of interest include glucose for diabetes management, cholesterol, and the like. In response to this growing importance of analyte detection, a variety of analyte detection protocols and devices for both clinical and home use have been developed.

One type of method that is employed for analyte detection is an electrochemical method. In such methods, a bodily fluid sample is placed into a sample-receiving chamber in an electrochemical cell that includes two electrodes, e.g., a counter and working electrode. The analyte is allowed to react with a redox reagent to form an oxidizable (or reducible) substance in an amount corresponding to the analyte concentration. The quantity of the oxidizable (or reducible) substance present is then estimated electrochemically and related to the amount of analyte present in the initial sample.

The electrochemical cell is typically present on a test strip which is configured to electrically connect the cell to an analyte measurement device. While current test strips are effective, the size of the test strips can directly impact the manufacturing costs. While it is desirable to provide test strips having a size that facilitates handling of the strip, increases in size will tend to increase manufacturing costs where there is an increased amount of material used to form the strip. Moreover, increasing the size of the test strip tends to decrease the quantity of strips produced per batch, thereby further increasing manufacturing costs. Accordingly, there is a need for improved electrochemical sensing apparatus and methods.

These and other embodiments, features and advantages will become apparent to those skilled in the art when taken with reference to the following more detailed description of various exemplary embodiments of the invention in conjunction with the accompanying drawings that are first briefly described.

### BRIEF DESCRIPTION OF THE DRAWINGS

The accompanying drawings, which are incorporated herein and constitute part of this specification, illustrate presently preferred embodiments of the invention, and, together with the general description given above and the detailed description given below, serve to explain features of the invention (wherein like numerals represent like elements).

FIG. 1A is an exploded view of the layers of an exemplary biosensor;

FIG. 1B is a perspective assembled view of the biosensor of FIG. 1A assembled;

FIG. 1C is a side view of the assembled biosensor of FIG. 1B;

FIG. 1D is a top view of the assembled biosensor of FIG. 1B;

FIG. 1E is a bottom view of the assembled biosensor of FIG. 1B;

FIG. 2A illustrates an inward side of the material of the first electrode including spacers and cut lines;

FIG. 2B illustrates a side view of the material of the first electrode of FIG. 2A;

FIG. 2C illustrates an inward side of the material of the second electrode;

FIG. 2D illustrates a side view of the material of the second electrode of FIG. 2C;

FIG. 2E illustrates a top view of the joined first and second electrodes after castellation;

FIG. 2F illustrates a side view of the joined castellated first and second electrodes of FIG. 2E;

FIG. 2G illustrates a bottom view of the joined castellated first and second electrodes;

FIG. 2H illustrates a cutting pattern of the castellated first and second electrodes of FIG. 2E used for singulation;

FIGS. 2I-2J illustrates a second cutting pattern of the cut and castellated first and second electrodes of FIG. 2G used for singulation; and

FIGS. 3A-3D illustrate a carrier for receiving an assembled sensor to form a test strip.

### MODES OF CARRYING OUT THE INVENTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present disclosure is defined solely by the claims. The features illustrated or described in connection with one exemplary embodiment may be combined with the features of other embodiments. Such modifications and variations are intended to be included within the scope of the present disclosure.

As used herein, the terms “patient” or “user” refer to any human or animal subject and are not intended to limit the systems or methods to human use, although use of the subject invention in a human patient represents a preferred embodiment.

The term “sample” means a volume of a liquid, solution or suspension, intended to be subjected to qualitative or quantitative determination of any of its properties, such as the presence or absence of a component, the concentration of a component, e.g., an analyte, etc. The embodiments of the present invention are applicable to human and animal samples of whole blood. Typical samples in the context of the present invention as described herein include blood, plasma, red blood cells, serum and suspensions thereof.

The term “about” as used in connection with a numerical value throughout the description and claims denotes an interval of accuracy, familiar and acceptable to a person skilled in the art. The interval governing this term is preferably ±10%. Unless specified, the terms described above are not intended to narrow the scope of the invention as described herein and according to the claims. The terms “top” and “base” as used herein are intended to serve as a reference for illustration purposes only, and that the actual position of the portions of the test strip will depend on its orientation.

The present invention generally provides a biosensor, such as an analytical test strip having disposed electrodes that are configured to communicate with an analyte measurement system, and more particularly a portable test meter. The test strip design is particularly advantageous in that the design is compact, while providing a large effective surface area for ease of handling. The smaller size of the electrochemical biosensor may reduce manufacturing costs, as less material is required to manufacture it.

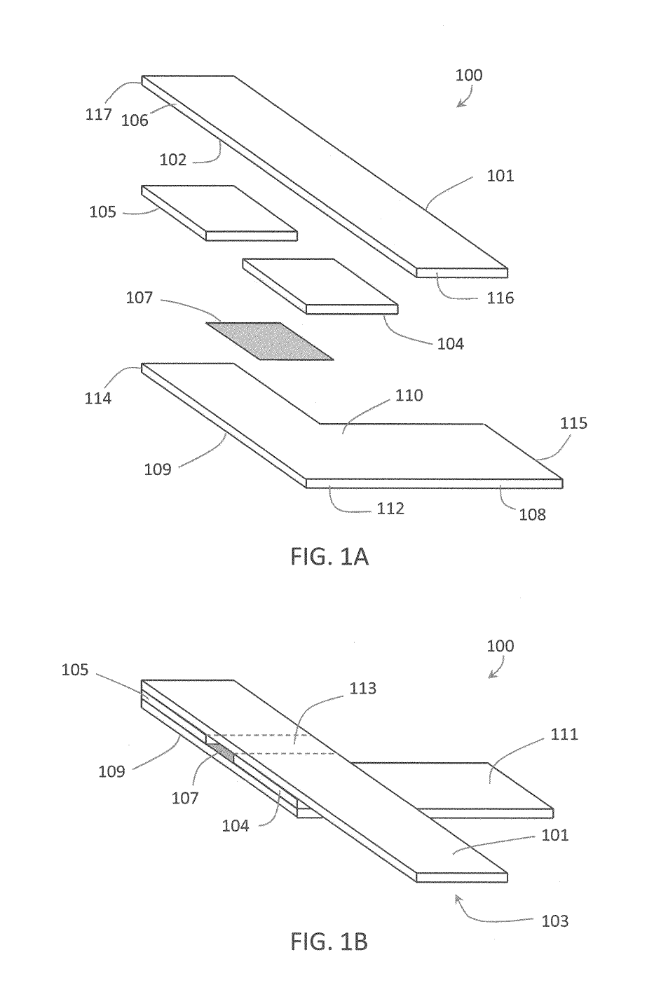

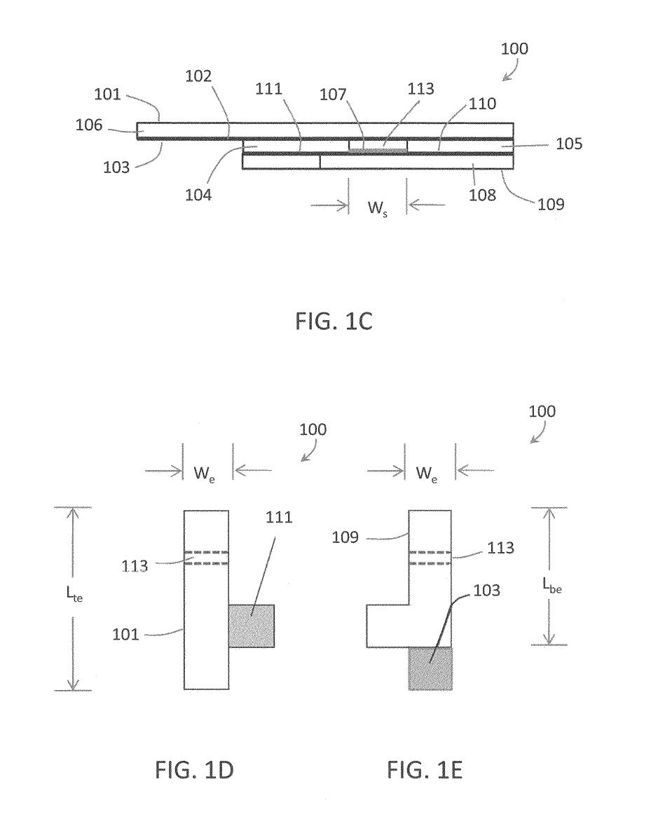

FIGS. 1A and 1B illustrate one exemplary embodiment of an analytical test strip 100. As shown, the test strip 100 generally includes a pair of electrodes, namely a top electrode 101 and a base electrode 109, each of the electrodes being defined by a planar construction including a substrate having a conductive layer and an insulating layer. The top electrode 101 is defined by a substantially rectangular planar construction and the base electrode 109 being defined by a substantially L-shaped planar configuration. A pair of spacers 104, 105 are sandwiched between a lower surface of the top electrode 101 and an upper surface of the base electrode 109, the spacers 104, 105 being axially spaced wherein opposing sidewalls of the spacers and the upper surface of the base electrode 109 and the lower surface of the top electrode 101 combine to define a sample cell or chamber 113, which functions as an electrochemical cell, as shown in FIG. 1B. A reagent layer 107 is disposed onto the upper surface of the base electrode 109 within the confines of the herein defined sample cell 113. A person skilled in the art will appreciate that the biosensor 100 can have various configurations other than those shown, and can include any combination of features disclosed herein and known in the art. Moreover, each test strip 100 can include a sample chamber 113 at various locations for measuring the same and/or different analytes in a sample.

The biosensor 100 can have various configurations, but is typically in the form of one or more rigid or semi-rigid layers having sufficient structural integrity to allow handling and connection to an analyte measurement system, as will be discussed in further detail below. The biosensor 100 may be formed from various materials, including plastic and other insulating materials. The material of the various layers, other than the reagent layer 107, typically is one that is insulating (non-conductive) and may be inert and/or electrochemically non-functional, where they do not readily corrode over time nor chemically react with a sample applied to the biosensor 100. The top electrode 100 includes a conductive material, or layer, 102 disposed on the bottom surface thereof (facing the base electrode 109). The base electrode 109 also includes a conductive material, or layer, 110 disposed on the upper surface thereof (facing the top electrode 101). The conductive layers 102, 110 should be resistant to corrosion wherein their conductivity does not change during storage of the biosensor 100.

In the assembled embodiment shown in FIG. 1B, the base electrode 109 of the test strip 100 has a generally elongate rectangular shape with an electrically conductive portion 111 formed on the base electrode 109 extending in a substantially orthogonal, or transverse, direction therefrom that may be referred to herein as a side tab electrical contact. The top and base electrodes 101, 109 may allow an analyte measurement system to engage the electrodes and measure an analyte concentration of a sample provided in sample chamber 113. Such a configuration facilitates connection to an analyte measurement device, as will be discussed further below.

The top and base electrodes 101, 109 include a substantially insulating and inert substrate, 106, 108, respectively, and have a conductive material disposed on one surface thereof 102, 110, respectively, to facilitate communication between electrodes of the electrochemical biosensor and an analyte measurement system or device. The electrically conducting layers 102, 110 can be formed from any conductive material, including inexpensive materials, such as aluminum, carbon, graphene, graphite, silver ink, tin oxide, indium oxide, copper, nickel, chromium and alloys thereof, and combinations thereof. However, precious metals that are conductive, such as palladium, platinum, indium tin oxide or gold, can optionally be used. The electrically conducting layers may be disposed on the entire inward facing surfaces of the top and base electrodes 101, 109, or they may terminate at a distance (e.g., 1 mm) from the edges of the electrodes 101, 109 but the particular locations of the electrically conducting layers should be configured to electrically couple the electrochemical biosensor to an analyte measurement system or device. In one exemplary embodiment, the entire portion or a substantial portion of the inwardly facing surfaces of the top and base electrodes 101, 109 are coated with the electrically conducting layers 102, 110 at a preselected thickness. As a result, each of the top and base electrodes 101, 109 includes an electrically conducting coating disposed thereon. Thus, when the electrochemical biosensor is assembled, as shown in FIG. 1B, the top electrode 101 will be positioned such that at least a portion of the inwardly facing conductive surface 102 of the top electrode 101 and the inwardly facing conductive surface 110 of the base electrode 109 are in facing relationship, i.e. “co-facial”, with one another. A person skilled in the art will appreciate that top and base electrodes 101, 109 can be manufactured to include separate layers such as an insulating layer 106, 108 adhered to a conductive metallic layer 102, 110, respectively, rather than forming a conductive coating on an insulating substrate.

To maintain electrical separation between the top and base conductive layers 102, 110, the biosensor 100 may further include a spacer layer, comprising proximal and distal spacers 104, 105, which may also be adhesive spacers for securing the top and base electrodes 101, 109, in a spaced relationship. The spacers 104, 105 can function to maintain the top and base electrodes 101, 109 at a distance apart from one another, thereby preventing electrical contact between the co-facial top and bottom electrically conducting layers 102, 110. The spacer layer may include double-sided adhesive spacers 104, 105 to adhere the top and base electrodes 101, 109 to one another. The spacers 104, 105 may be formed from a variety of materials, including a material with adhesive properties, or the spacers 104, 105 can include a separate adhesive used to attach the spacers 104, 105 to the electrodes 101, 109. Non-limiting examples of ways in which adhesives can be incorporated into the various biosensor assemblies of the present disclosure can be found in U.S. Pat. No. 8,221,994 of Chatelier et al., entitled “Adhesive Compositions for Use in an Immunosensor”, the contents of which is incorporated by reference as if fully set forth herein in its entirety.

The spacers 104, 105 may have various shapes and sizes and can be positioned in various positions between the top and base electrodes 101, 109. In the embodiment shown in FIGS. 1A-1B, spacers 104, 105 are spatially separated by a distance Wₛ (FIG. 1C) to define sidewalls of the sample chamber 113. A person skilled in the art will appreciate that the location of the spacers, and the sample chamber defined thereby, can vary. Similarly, the biosensor can also include electrical contact pads 103, 111 located anywhere on the conductive layers 102, 110, respectively, for coupling to an analyte measurement system or device. In the illustrated embodiment, the electrical contact pads 103, 111, are configured to establish a connection between the top and bottom electrodes 101, 109, respectively, of the biosensor 100 and an analyte measurement system or device.

As best shown in FIGS. 1D-1E, the biosensor may be considered to include a main body having top and base electrode contacts extending therefrom. The main body is defined by the trilaminate structure formed above and between first and second terminal ends 112, 114 of the base electrode and includes the spacers 104, 105 and the portion of the top electrode directly above the base electrode. The main body is generally defined by a width Wₑ of the biosensor 100 and a length L_{be} of the bottom electrode 109 including the layers of the biosensor 100 within the length L_{be} and width Wₑ. The top electrode contact pad 103 extends in a first direction from the main body while the base electrode contact pad 111 extends from the main body in a second direction substantially transverse to the main body, or orthogonal to the first direction. The extensions from the main body of both electrical contact pads 103, 111 serve to expose the contact pads 103, 111 of the electrically conducting layers 102, 110 on the inwardly facing surfaces of the top and base electrodes 101, 109. A person skilled in the art will appreciate that the electrical contacts can have a variety of configurations other than those illustrated.

The configuration of the electrical contact pads 103, 111 allows an analyte measurement system or device to electrically contact the electrodes 101, 109. The biosensor 100 can be configured to couple to a variety of analyte measurement systems and devices as explained below.

In one embodiment, the biosensor 100 may include top and base electrodes 101, 109 and a reagent film, or layer, 107 on the electrically conductive layer 110 of the base electrode between the spacers 104, 105. The reagent layer 107 reacts with an analyte in a fluid sample provided in the sample chamber 113 by a user of the biosensor. The top and base electrodes may be configured in any suitable configuration in an opposed spaced apart relationship for receiving a sample. The illustrated reagent film 107 may be disposed on either of the top or base electrodes 101, 109 and between the spacers 104, 105 and within the chamber 113 for coming into contact, and reacting, with an analyte in an applied sample. A person skilled in the art will appreciate that the electrochemical biosensor 100 may have a variety of configurations, including having other electrode configurations, such as co-planar electrodes.

In the illustrated embodiment, the spacers 104, 105 each have a generally square or rectangular shape. The spacers 104, 105 may be formed from various materials, but in an exemplary embodiment they are formed from a material having a small coefficient of thermal expansion such that the spacers do not adversely affect the volume of the sample chamber 113. As shown in FIG. 1A, an inwardly facing surface of the top electrode 101 and an opposing inwardly facing surface of the base electrode 109 each carry a conducting layer 102, 110. The electrodes 101, 109 may be formed from conducting layers 102, 110 including gold, palladium, carbon, silver, platinum, tin oxide, iridium, indium, graphene and combinations thereof (e.g., indium doped tin oxide) deposited, adhered, or coated on the insulating layers 106, 108. The conductive layer may be deposited onto the insulating layers 106, 108 by various processes, such as sputtering, electroless plating, thermal evaporation and screen printing. In one exemplary embodiment, the reagent-free electrode, e.g., the top electrode 101, is a sputtered gold electrode, and the electrode containing the reagent 107, e.g., the base electrode 109, is a sputtered palladium electrode. As discussed in further detail below, in use one of the electrodes can function as a working electrode and the other electrode can function as the counter/reference electrode.

When the electrochemical biosensor 100 is assembled, the top and base electrodes may be held together at a spaced distance apart by one or more of the spacers 104, 105 which have a generally rectangular configuration with a width that can be substantially equal to a width Wₑ (FIG. 1D) of the electrodes 101, 109 and a length that is significantly less than either of the electrodes 101 (Lₜₑ) or 109 (L_{be}). However, the shape and size of the spacers 104, 105 can vary significantly.

As shown in FIG. 1B, the proximal spacer 104 is positioned adjacent to a first terminal end 112 of the base electrode 109, and the distal spacer 105 is positioned adjacent to a second terminal end 114 of the base electrode 109 such that a space or gap 113 is defined between the proximal and distal spacers. The second terminal end 116 of the top electrode 101 can be positioned in substantial alignment with the first terminal end 112 of the base electrode 109. As explained above, the first terminal end 116 of the top electrode extends a distance beyond the main body, i.e., beyond the first terminal end 112 of the base electrode 109 in a first direction. A third terminal end 115 of the base electrode 109 extends from the main body transversely in a second direction orthogonal to the first direction that the first terminal end 116 of the top electrode insulating layer extends from the main body. The base electrode thus comprises a right angle, or an L shape. These extensions from the main body expose inwardly facing portions of the conducting layers 102, 110 of each of the top and base electrodes 101, 109 which define the electrical contact pads 103, 111 of the biosensor 100. A person skilled in the art will appreciate that the particular configuration, including the shape, orientation, and location of the spacers and the insulating layers relative to one another can vary.

As indicated above, the spacers 104, 105 and the electrodes 101, 109 generally define a space or gap, also referred to as a window, therebetween which forms an electrochemical cavity or sample chamber 113 for receiving a sample. In particular, the top and base electrodes 101, 109 define the top and bottom of the sample chamber 113 and the spacers 104, 105 define the sides of the sample chamber 113. The gap between the spacers 104, 105 will result in an opening or inlet extending into the sample chamber 113. The sample can thus be loaded through the opening or inlet. In one exemplary embodiment, the volume of the sample chamber can range from about 0.1 microliters to about 5 microliters, preferably about 0.2 microliters to about 3 microliters, and more preferably about 0.2 microliters to about 0.4 microliter. To provide the small volume, the gap between the spacers 104, 105 have an area ranging from about 0.005 cm² to about 0.2 cm², preferably about 0.0075 cm² to about 0.15 cm², and more preferably about 0.01 cm² to about 0.08 cm², and the thickness of the spacers 104, 105 can range from about 1 micron to 500 microns, and more preferably about 10 microns to 400 microns, and more preferably about 40 microns to 200 microns, and even more preferably about 50 microns to 150 microns. As will be appreciated by those skilled in the art, the volume of the sample chamber 113, the area of the gap between the spacers 104, 105, and the distance between the electrodes 101, 109 can vary significantly.

As further illustrated, the sample chamber 113 may also include a reagent film or layer 107 disposed on at least one of the electrodes, e.g., the base electrode 109 as illustrated. Alternatively, the reagent layer can be disposed on multiple faces of the sample chamber 113. The reagent layer 107 can be formed from various materials, including various mediators and/or enzymes. Suitable mediators include, by way of non-limiting example, ferricyanide, ferrocene, ferrocene derivatives, osmium bipyridyl complexes, and quinone derivatives. Suitable enzymes include, by way of non-limiting example, glucose oxidase, glucose dehydrogenase (GDH) based onpyrroloquinoline quinone (PQQ) co-factor, GDH based on nicotinamide adenine dinucleotide co-factor, and FAD-based GDH. One exemplary reagent formulation, which would be suitable for making the reagent layer 107, is described in U.S. Pat. No. 7,291,256, entitled “Method of Manufacturing a Sterilized and Calibrated Biosensor-Based Medical Device,” the entirety of which is hereby incorporated as if fully set forth herein by reference. The reagent layer 107 can be formed using various processes, such as slot coating, dispensing from the end of a tube, ink jetting, and screen printing. While not discussed in detail, a person skilled in the art will also appreciate that the various electrochemical modules disclosed herein can also contain a buffer, a wetting agent, and/or a stabilizer for the biochemical component.

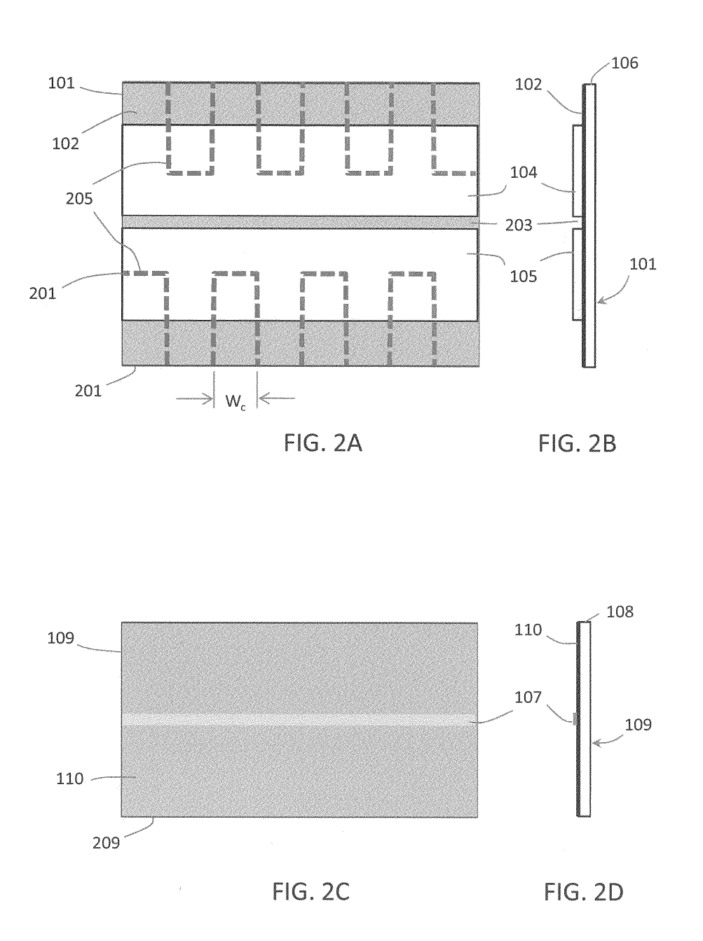

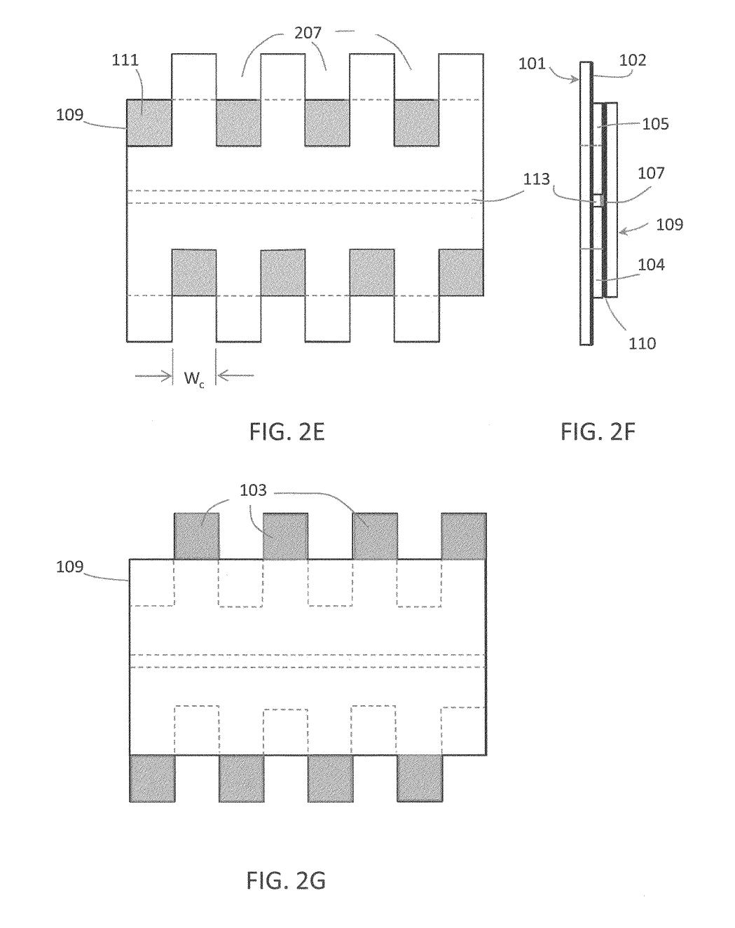

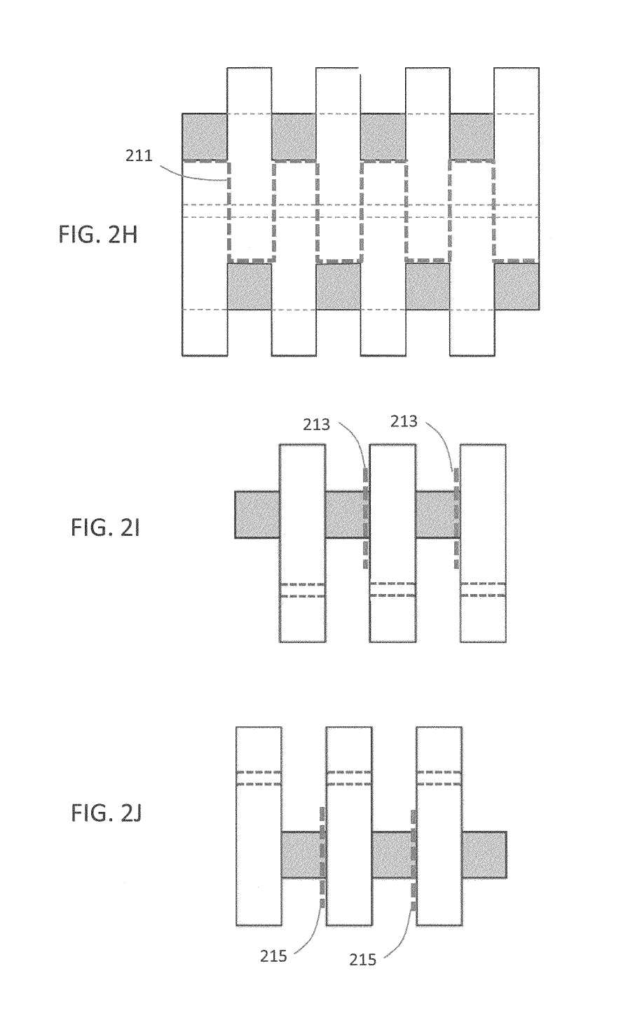

With reference to FIGS. 2A-2B, and according to one embodiment, the material used for fabricating the top electrode 101 can be formed as a continuous web 201, generally rectangular in shape having two opposite parallel edges, and comprising an insulator layer 106 and a conductive layer 102 deposited thereon. The pair of spacers 104, 105 are deposited, laminated, or adhered onto the conductive layer 102 and are separated by a gap 203 which eventually forms the sample chamber 113. The spacers may be deposited in parallel to form a straight line gap therebetween.

With reference to FIG. 2C, the material used to form the base electrode 109 is also formed as a continuous web 209 of material comprising an insulating layer 108 and a conductive layer 110 deposited thereon. A strip of the sample chamber reagent 107 is deposited in generally a straight line along a central axis of the web, and dried. The sample chamber reagent is deposited along the conductive layer 110 such that it will align with the gap 203 between the spacers 104, 105 when the continuous webs 201 and 209, together with the spacers 104, 105 and the reagent layer 107 are joined together. The top electrode web 201 comprises a greater width than the base electrode web 209 as can be seen when these webs are laminated or overlaid, as shown in FIG. 2E. Prior to overlaying and joining the webs 201, 209, the continuous web 201 is castellated together with the spacers 104, 105 along the cut lines 205 as seen in FIG. 2A. The castellated features 207 (FIG. 2E) on opposite edges of the web 201 are offset by the width of the castellation W_{c} so that the singulation steps, as described below, will result in the desired shape of the individual biosensors 100 formed by the singulation.

The laminating step forms a trilaminate structure as seen in FIG. 2F comprising, in general, the top and base electrodes 101, 109 (webs) and spacers 104, 105, therebetween. However, this trilaminate structure exposes a portion of the base electrode 109 that will eventually form the base electrode contact pads 111 by virtue of the top electrode castellation features 207, as seen in FIG. 2E. A portion of the upper electrode's conductive layer 102 is exposed by virtue of the narrower width of the base electrode 109, which exposed portions will eventually form the top electrode contact pads 103, as seen in FIG. 2G. The trilaminate web is then subject to cutting along the cut line 211 illustrated in FIG. 2H to create two separate singulated webs as shown in FIGS. 2I and 2J. These singulated webs, in turn, may be further separated along cut lines 213, 215, to yield singulated individual co-facial biosensors with exposed and easily accessible electrical contact pads 103, 111 as illustrated in FIGS. 1D and 1E.

It should be noted that the fabrication steps just described may be modified in various combinations as is well known to those skilled in the art. For example, the base electrode material may be used to form the wider, castellated web, and a non-castellated web used to form the top electrode, effectively reversing the exposed contact points within the system. The reagent layer may be applied, as necessary, to the top electrode conducting layer in that instance. The fabrication steps just described may be appropriately sequenced in various combinations and is considered to be within the scope of the present disclosure. One advantage of this approach is that it makes use of an interlocking sensor web design that, when cut, forms two continuous sensor webs without wasting fabrication materials. This may be achieved by ensuring that the biosensor reagent strip is centrally placed on the web, so that when the two sensor webs are singulated by cutting, then two identical functioning webs of sensors are created.

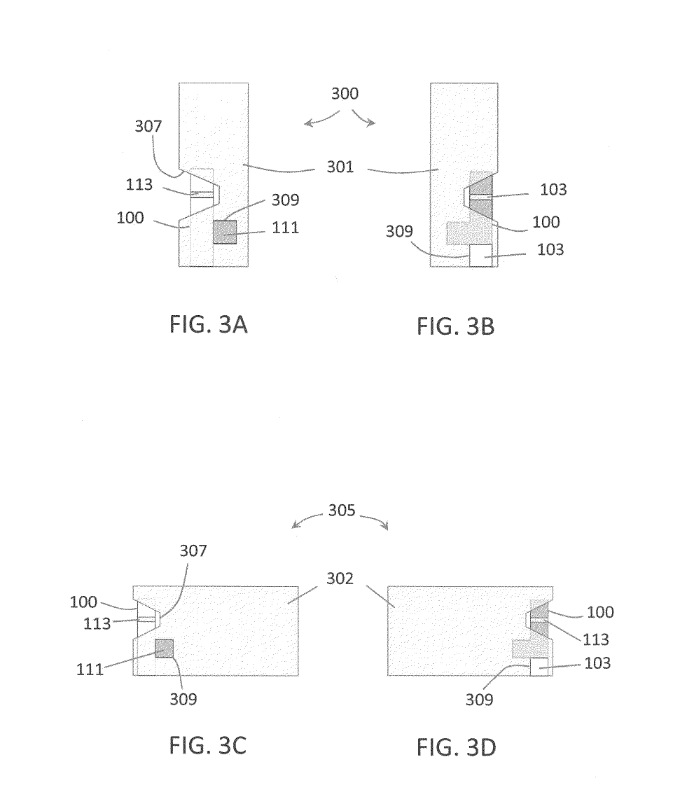

With reference to FIGS. 3A-3D there are illustrated examples of inert carriers 301, 302, that can be attached to the biosensor 100 embodiments described herein to form a test strip 300, 305 that may be inserted into an analyte measurement system or device for measuring an analyte concentration of a sample provided by a user. As shown, the test strip 300, 305 generally includes a carrier 301, 302 and an electrochemical biosensor 100 is mounted in the carrier 301, 302, as shown in FIGS. 3A-3D. In general, the carrier 301 has dimensions that are greater than the biosensor 100, such that the carrier 301, 302 serves as a support to facilitate handling of the biosensor 100. A person skilled in the art will appreciate that the test strip 300, 305 can have various configurations other than those shown, and can include any combination of features disclosed herein and known in the art. Moreover, each test strip 300, 305 may include any number of electrochemical biosensors at various locations on the carrier for measuring the same and/or different analytes in a fluid sample. The carrier 301, 302, may be in the form of one or more rigid or semi-rigid substrates having sufficient structural integrity to support the electrochemical biosensor 100 and to allow handling and connection to an analyte measurement device. The carrier can be formed from various materials, including plastic or cardboard materials. In an exemplary embodiment, materials that do not shed or that exhibit relatively low shedding of fibers are preferred. The substrate material typically is one that is non-conductive. The carrier material can also have any thermal coefficient of expansion, including a low thermal coefficient of expansion, as changes in the volume of the material during use will not have any effect on performance. In addition, the carrier materials may be inert and/or electrochemically non-functional, where they do not readily corrode over time nor chemically react with biosensor 100 material.

The shape of the carrier 301, 302 can also vary. In the embodiments shown in FIGS. 3A-3D, the carrier 301, 302 has a generally elongate rectangular shape. The carrier 20 can be formed from separate first and second layers that are in facing relationship with one another, such as an envelope. The first and second layers of the carrier allow an electrochemical biosensor 100 to be inserted and secured therebetween. The non-conducting substrate of the carrier can be cut at one edge in order to form an opening 307 to facilitate insertion of the biosensor 100 or to provide access to the inlet of the sample chamber 113 of the electrochemical biosensor 100 when the biosensor 100 is disposed in the carrier 301, 302.

The carrier 301, 302 also includes windows 309 to provide access and to facilitate communication between contact pads 103, 111 of the biosensor 100 and an analyte measurement system or device when the test strip 300, 305 is inserted therein. The quantity of edge cut openings 307 and the location of each opening 307 can vary depending on the intended use, for example, whether more than one biosensor 100 will be present in a carrier 301, 302. In the illustrated embodiments, the opening 307 is positioned along a perimeter of the carrier 301, 302. While not shown, the opening 307 can alternatively be positioned along any edge of the carrier 301, 302. The interior surfaces of the first and second layers of the carrier 301, 302 may include an adhesive to secure the biosensor therewithin.

### PARTS LIST FOR FIGS. 1A-3D

100 biosensor
101 top electrode
102 top electrode conducting layer
103 top electrode contact pad
104 spacer—proximal
105 spacer—distal
106 insulating layer
107 reagent layer
108 insulating layer
109 base electrode
110 base electrode conducting layer
111 base electrode contact pad
112 base electrode first terminal end
113 sample chamber
114 base electrode second terminal end
115 base electrode third terminal end
116 top electrode first terminal end
117 top electrode second terminal end
201 top electrode continuous web
203 gap between spacers
205 cut lines
207 castellation features
209 base electrode continuous web
211 cut lines
213 cut lines
215 cut lines
300 test strip
301 carrier
302 carrier
305 test strip
307 carrier opening
309 carrier window

While the invention has been described in terms of particular variations and illustrative figures, those of ordinary skill in the art will recognize that the invention is not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. Therefore, to the extent there are variations of the invention, which are within the spirit of the disclosure or equivalent to the inventions found in the claims, it is the intent that this patent will cover those variations as well.

What is claimed is:

1. A biosensor comprising:
a main body;
a first electrode;
a second electrode, each of the first and second electrodes having a conductive surface;
a pair of spacers disposed between the first and second electrodes and maintaining the first and second electrodes in a spaced apart relationship; and
and wherein a portion of the first electrode is cantilevered from the main body in a first direction and a portion of the second electrode is cantilevered from the main body in a second direction and wherein the first and second directions are substantially transverse to one another.

2. The biosensor of claim 1, wherein the conductive surfaces of the first and second electrodes face one another.

3. The biosensor of claim 1, wherein the pair of spacers define a first pair of walls of a sample chamber of the biosensor.

4. The biosensor of claim 3, wherein the first and second electrodes define a second pair of walls of the sample chamber.

5. The biosensor of claim 3, wherein at least one of the second pair of walls includes a reagent deposited thereon, and wherein the sample chamber is configured to receive a fluid sample therein, to generate a reaction between the fluid sample and the reagent, and to complete an electrical circuit between the first and second electrodes via the reacted fluid sample.

6. The biosensor of claim 5, wherein a portion of the first conducting surface comprises a first electrical contact pad for engaging a first electrical contact of an analyte meter when the biosensor is inserted therein.

7. The biosensor of claim 6, wherein a portion of the second conducting surface comprises a second electrical contact pad for engaging a second electrical contact of the analyte meter when the biosensor is inserted therein.

8. The biosensor of claim 1, wherein the first electrode comprises a first insulating substrate carrying the first conducting surface and a second insulating substrate carrying the second conducting surface.

9. A biosensor comprising:
a first electrode;
a second electrode, each of the first and second electrodes having an insulating layer and a conducting layer wherein one of the electrodes is defined by a substantially elongated rectangular shape and the other of said electrodes is defined by a substantially L shaped configuration;
a pair of spacers disposed between the first and second electrodes and abutting each of the conducting layers to maintain a spaced relationship therebetween; and
wherein a portion of the first electrode overlaps a portion of the second electrode such that a first terminal end of the first electrode extends a distance beyond a first terminal end of the second electrode to expose a portion of the first conducting layer of the first electrode.

10. The biosensor of claim 9, wherein the exposed portion of the first conducting layer comprises a first contact pad of the biosensor.

11. The biosensor of claim 10, wherein a non-overlapping portion of the second electrode exposes a portion of the second conducting layer, and wherein the exposed portion of the second conducting layer comprises a second contact pad of the biosensor.

12. The biosensor of claim 11, wherein the pair of spacers are separated by a gap, a portion of the first and second conducting layers face each other across the gap, and wherein said portions of the first and second conducting layers and said spacers define a sample chamber of the biosensor.

13. The biosensor of claim 12, wherein at least one of said portions of the first and second conducting layers comprise a reagent layer thereon for reacting with a sample applied to the sample chamber to form an electrochemical cell.

14. A method of making a biosensor, the method comprising:
forming a first electrode web comprising a first insulating layer and a first conducting layer;
forming a second electrode web comprising a second insulating layer and a second conducting layer;
forming a pair of spacers on the first conducting layer;
forming a reagent layer on the second conducting layer;
castellating two edges of the first electrode web and the spacers thereon;
joining the second electrode web against the pair of spacers on the first electrode web and aligning the reagent layer with a gap between the pair of spacers; and
singulating the joined first and second electrode webs together with the spacers and the reagent layer thereon to form a plurality of singulated webs each comprising singulated first and second electrodes, spacers, and a reagent layer.

15. The method of claim 14, further comprising singulating at least one of the plurality of singulated webs to form a plurality of individual biosensors each comprising a first and a second electrode, a pair of spacers, and a reagent layer.

16. The method of claim 14, further comprising forming an adhesive layer on a first surface of each of the pair of spacers, the first surface facing away from the first conducting layer.

17. The method of claim 14, further comprising forming the pair of spacers on the first conducting layer in parallel such that the gap between the pair of spacers forms a straight line.

18. The method of claim 17, further comprising forming the reagent layer on the second conducting layer in a straight line that coincides with the gap between the pair of spacers.

19. The method of claim 14, wherein the two castellated edges of the first electrode web are opposite and parallel edges.

20. The method of claim 19, wherein the step of castellating forms castellation features on the opposite and parallel edges of the first electrode web that are offset from each other by a width of the castellation features.
